(19) United States
(12) Patent Application Publication
(10) Pub. No.: US 2014/0146048 A1
(43) Pub. Date: May 29, 2014

(54) STANDARDIZED GEOMETRIC AND PHYSIOLOGIC SPACE FOR VISUAL AND QUANTITATIVE EVALUATION OF TUMOR MRI CHARACTERISTICS

(71) Applicants: Xu Feng, Seattle, WA (US); Bartholomew Patrick Keogh, Seattle, WA (US)

(72) Inventors: Xu Feng, Seattle, WA (US); Bartholomew Patrick Keogh, Seattle, WA (US)

(21) Appl. No.: 14/088,285

(22) Filed: Nov. 22, 2013

Related U.S. Application Data
(60) Provisional application No. 61/729,493, filed on Nov. 23, 2012.

(51) Int. Cl. G06T 17/00; A61B 5/055

(52) U.S. Cl. CPC G06T 17/00; A61B 5/055
USPC 345/424

(57) ABSTRACT

A system and method for displaying MRI parameters measured from a tissue sample such a tumor. A computer system receives MRI data and determines a number of parameters including, a normalized cerebral blood volume (nCBV), a contrast transfer rate constant (kTrans) and apparent diffusion coefficient (ADC) calculated from Bo and B1000 MRI images for data points in the MRI data. These parameters are plotted onto a three-dimensional graph of voxels where the data in the MRI are mapped to a normalized radial distance and angle in the plot. The parameters nCBV, kTrans and 1/ADC are mapped to a color of the voxel and to height of a voxel in the +Z and −Z axes of the three dimensional plot.

### CROSS-REFERENCE TO RELATED APPLICATION(S)

The present application claims the benefit of, and priority to, U.S. Provisional Patent Application No. 61/729,493 filed Nov. 23, 2012, which is herein incorporated by reference in its entirety.

### TECHNICAL FIELD

The disclosed technology relates to medical imaging systems and in particular to systems for analyzing and displaying MRI data from tumors.

### SUMMARY

A computer system in accordance with one embodiment of the disclosed technology receives MRI image data and calculates a number of parameters including, normalized cerebral blood volume (nCBV), contrast transfer rate constant (kTrans) and apparent diffusion coefficient (ADC) calculated from Bo and B1000 MRI images as source MRI data for subsequent analysis. These parameters are plotted onto a five-dimensional graph (x, y, +z, −z, color) by voxels where the physiologic data across multiple MRI images are mapped to a normalized radial distance and angle in the 5 dimensional space. The parameters nCBV, kTrans and 1/ADC are mapped to specific dimensions in this multidimensional representation. In one particular embodiment, the nCBV parameter is mapped to a height of a voxel in the +Z axis, the kTrans parameter is mapped to the height of a voxel in the −Z axis and the parameter 1/ADC is mapped to a color of a voxel.

### BRIEF DESCRIPTION OF THE DRAWINGS

FIGS. 1A-C show a low grade astrocytic tumor. Evident from the standardized space representation is the low nCBV, kTrans and variable (but relatively low) cellularity as measured by 1/ADC.

FIGS. 2A-C show a low grade neuroglial tumor. Evident from the standardized space representation is the low nCBV, kTrans and quite low cellularity as measured by 1/ADC. The difference between these two low grade lesions is evident based on the expression in the standardized graphing space.

FIGS. 3A-C show a high grade astrocytic tumor. The difference between this tumor and the low grade tumors (FIGS. 1A-C, 2A-C) is evident as high nCBV, high kTrans and high cellularity.

FIGS. 4A-C show radiation necrosis. This is radiographically indistinguishable from high grade astrocytic tumors or recurrence based on standard anatomic imaging. In the standardized space, this lesion appears immediately different from the high grade astrocytoma shown in FIGS. 3A-C.

FIGS. 5A-C show a pseudo-progression.

FIGS. 6A-C show a true progression.

FIGS. 7A-C show a B-cell lymphoma. The high cellularity is reflected in the low (red) ADC values while the modest elevation in nCBV is related to absent microvascular proliferation (not a feature of lymphoma).

FIGS. 8A-C show the application of the visual representation and analysis method here described to two low grade astrocytic tumors (FIGS. 8A and 8B), with average of the tumors shown in FIG. 8C.

FIGS. 9A-C show the application of the visual representation and analysis method here described to two high grade astrocytic tumors (FIGS. 9A and 9B), with average of the tumors shown in FIG. 9C.

FIGS. 10A-C show the application of the visual representation & analysis method here described to two examples of tumor recurrence (FIG. 10A and 10B), with average of the tumors shown in FIG. 10C.

FIGS. 11A-C show the application of the visual representation & analysis method here described to two examples of radiation necrosis (A and B), with average of the tumors shown in C.

FIG. 12 illustrates a medical imaging system in accordance with one embodiment of the disclosed technology.

FIG. 13 shows a region of interest (ROI) drawn by an operator to locate a tumor in a specific MRI slice.

### DETAILED DESCRIPTION

As will be explained in further detail below, the technology described herein relates to a medical imaging system that virtualizes and allows single image display of a number of parameters derived from MRI images of tissue and in particular to MRI images of tumors. In one embodiment, a programmed processor executes instructions to determine a number of parameters for tissue in a region of interest in an MRI image. The computer system then creates a multi-dimensional (in this case 5 dimensional) plot of the measured parameters that can be used by a physician to infer qualities or characteristics about a tumor being imaged. In one embodiment, the parameters are plotted in a normalized space so that comparisons between different tumors can be made. In one particular embodiment, the measured or determined MRI parameters include, a normalized cerebral blood volume (nCBV), a contrast transfer rate constant (kTrans) and apparent diffusion coefficient (ADC) calculated from Bo and B1000 MRI images for data points in the MRI data. Voxels in the three-dimensional plot that correspond to data points in the MRI image are encoded with these parameter values. These parameters are plotted for points in the MRI data using the positive Z axis region and the negative Z axis region of the three-dimensional plot. In addition, the color of the voxels is used to represent one of the parameters. The shapes and colors of the plots are indicative of tumor characteristics such as the grade of a tumor (i.e. its aggressiveness).

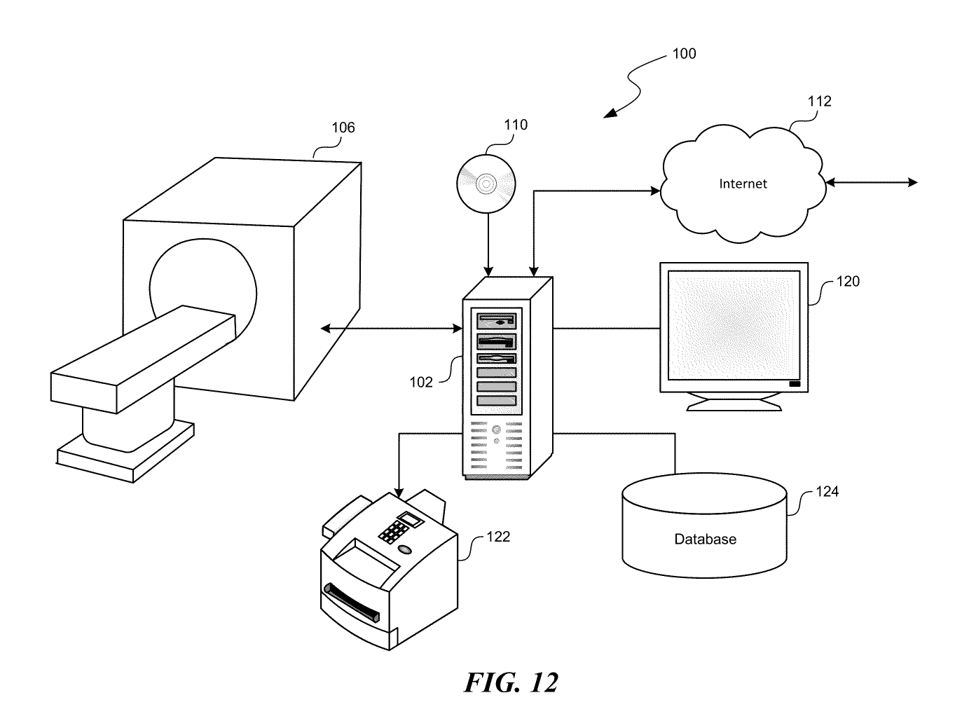

As shown in FIG. 12, a medical imaging system 100 includes a computer system 102 having one or more processors (not shown) that are configured to execute a program to produce a plot of MRI parameters measured from a tissue sample. In one embodiment, the computer system 102 is coupled to an MRI machine 106 that images a patient. Data from the MRI machine is received by the computer system 102 and analyzed by the computer to determine the parameters to be plotted. The instructions for the processor can be received on a non-transitory, computer readable media 110 (CD, hard drive, flash drive, solid state memory, remote server or the like). Alternatively, the instructions can be received over a computer network such as the internet 112.

The plot of the parameters is displayed for a clinician, radiologist or other user on a video display. Alternatively the plots can be printed on a printer 122. The computed parameters and/or plots can also be stored in a database 124 for later retrieval and analysis or transmitted to one or more remote computers via the internet 112.

As will be understood by those of ordinary skill in the art, intrinsic to current medical practice is the visualization of internal anatomic structures, allowing identification of abnormal tissue with varying malignant potential. This anatomic information has transformed medicine, allowing pre-clinical identification of neoplastic processes. The definition of response to therapy has also been redefined, with imaging identifying early recurrent cancers—acting as a surrogate for clinical response, before clinically evident patient functional loss (i.e. deterioration of clinical condition).

The evolution of tumor imaging has involved the addition of data that is not purely anatomic. Multiple kinds of physiologic data are now routinely acquired and utilized. The relationship between the physiologic and imaging data is most often presented as an “overlay” where the physiologic metric is geometrically aligned with anatomy—often colored and at some level of transparency. While this method is intuitive, it does not scale well as multiple physiologic metrics are considered. The result, in tumor imaging, has been proliferation of multiple “parametric maps” each displaying one variable across many images. The assimilation of this information by the user is challenging.

The variety of cell types from which tumors may derive, producing masses covering a range of characteristics with respect to size, shape and imaging metrics further complicates the unification of multiple kinds of mixed anatomic and physiologic data into a single cohesive “picture” of the tumor. The possibility of effective and efficient quantitative, and qualitative, comparisons between tumors requires reduction of tumor “dimensionality” to a common set of variables and scale.

Tumors, like any complex structure, can be approximated by some set of parameters that model the behavior of interest—analogous to principal components analysis. To compare biological entities of different geometric sizes and biological attributes, a set of relevant parameters is first chosen. In one embodiment, significant biological characteristics are approximated with a single dimension. The spatial geometry of the mass is simplified by assuming radial symmetry of the tumor—reducing complex geometries into two dimensions of distance from tumor centroid and angular distance from a defined direction. This assumption is tacitly part of many published reports, where multiple biopsies from the periphery, center and mid-aspect of a tumor (or across multiple tumors) are compared.

Biological behavior is significantly determined by non-geometric characteristics of the tumor; cellularity, vascularity, capillary density, capillary integrity, etc. In the embodiment described, a subset of these aspects are simplified into single dimensions. Apparent diffusion coefficient (ADC) is calculated from the Bo and B1000 images—low values are associated with high cellularity. Cerebral blood volume is calculated by consideration of the first pass curve (Dynamic Susceptibility Contrast (DSC)) and reflects micro-vascular surface area. Capillary integrity has also been shown to reflect tumor behavior and is measured by the contrast transfer rate constant (kTrans).

Dynamic Susceptibility Contrast imaging (DSC) is also often referred to as “perfusion imaging” and has been widely used in stroke care. In essence, DSC involves the rapid acquisition of sequential images at a given location(s) as the contrast bolus moves through the brain, the “first pass”. From the first pass curve, a number of physical quantities may be derived, including; cerebral blood volume (CBV), cerebral blood flow (CBF) and meant transit time (MTT).

The application of DSC to tumor imaging is a more complex undertaking than its' utilization in stroke, which is generally well implemented by any time derivative (e.g. MTT). Of the metrics derived from DSC, CBV has proven to be the most useful in evaluating tumors and treatment effect. The histopathological substrate reflected in CBV appears to be microvascular density. CBV values are most often normalized to contralateral tissue (nCBV)—in most cases normal contralateral white matter is used.

Essential considerations in the application of DSC in tumor imaging include leakage correction (mathematical or by contrast pre-load), arterial deconvolution (versus simpler mathematical techniques), choice of pharmacokinetic model the time course and dosing used for pre-load and consistent choice of the “index” tissue for normalization.

Application of DSC to tumor imaging has shown to be accurate in distinguishing low from high grade tumors with CBV as the most useful metric. Most interestingly, in both histologically low and high grade tumors, nCBV appears to provide additional stratification (independent of histopathology), identifying both low and high grade tumors that behave more aggressively. Notably, low-grade oligodendrogliomas may have elevated rCBV without the same implications of aggressive behavior due to the increased capillary density in all grades of this histopathologic subset.

Purely numerical approaches to this problem have been based on discriminant (and other) analysis. These approaches are mathematically rigorous, but lack the geometric relational component that is intrinsic to visuospatial representations. Given that information from imaging is applied by physicians and other caregivers, an approach that utilizes the intrinsic human capacity for understanding spatial relationships and patterns has advantages over purely numerical approaches.

The approach described here formally utilizes the inherent symmetry of a tumor to co-register multiple tumor voxels and physiological parameters that are important in tumor biology, into a standardized, partially geometric space.

Moreover, this approach preserves, in part, the relationship between tumor geometry and parametric value distribution, which is lost in purely mathematical approaches. Inherent in discriminant type approaches is dichotimazation of the data—a situation that often does not exist in clinical practice, where a range of differential considerations are possible.

The disclosed technology condenses a large volume of information into a single image, making evident differences and similarities between tumors that may not otherwise be appreciated on multiple distinct parametric maps. This approach also allows direct comparison between tumors of dissimilar size, shape and geometry—and may be utilized across tumors from multiple tissue types (lung, colon, breast, etc.) although the approach is developed in the context of intracranial tumors.

The preferred parameters:

DSC Measurements:

Standardization is performed relative to mixed white and grey matter by segmentation based on histogram analysis of raw CBV data. High values (corresponding to vessels) were excluded. Normalization excluded tumor volume and utilized the lower peak mean as the index.

DCE Measurements:

kTrans values are determined as absolute values, utilizing an arterial input function. No relative scaling is used. Standard software and mathematical techniques are used.

ADC Measurements:

ADC values were determined in standard fashion from Bo and B1000 images.

Tumor Segmentation:

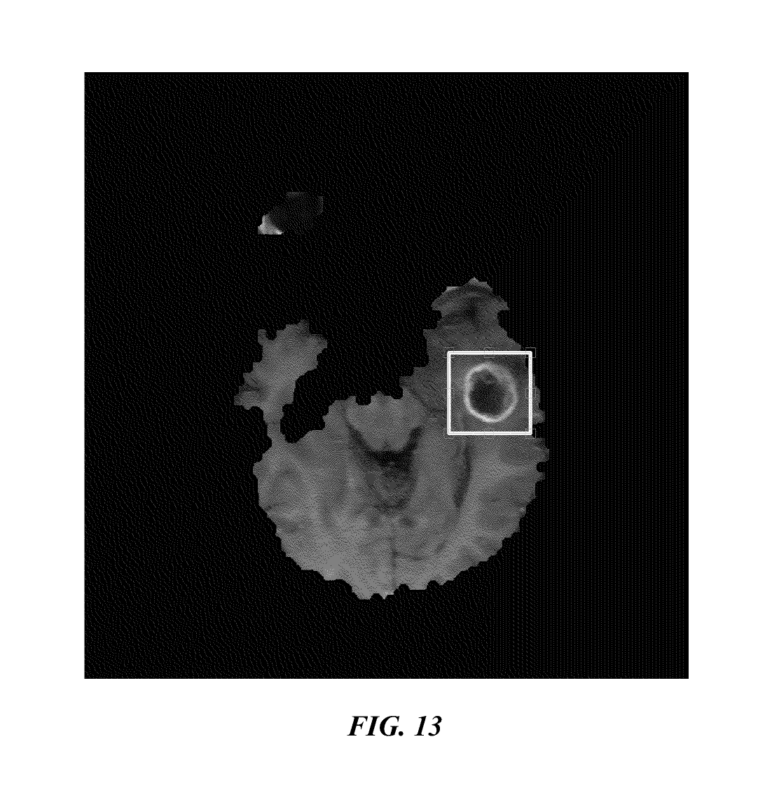

Semi-automated tumor segmentation is carried out by an operator first identifying the general location of the tumor on images to be used as the “anatomic basis”. Tumor margin is then identified automatically on T1 post contrast (enhancing tumors) or T2 weighted imaging. The operator specifies the adjacent slices of images which include the tumor and decides the best slice to draw a rectangle ROI. One example is shown as FIG. 13. The ROI is used to reduce the computation of segmentation algorithm. The threshold values are automatically calculated based on each slice. Only one ROI needs to be selected and the segmentation will be applied in all the adjacent slices.

Geometric Distance:

In one embodiment, a tumor centroid is determined by approximating intersection of lines spanning the enhancing or T2 tumor margins. For an odd number of slices with tumor, the centroid is in the center slice. For an even number of slices with a tumor, the centroid is calculated by the center of the centermost two slices. In a sense this is the spatial equivalent of the Mahalanobis distance in discriminant analysis. Geometric distance to any given voxel was calculated based on the normalized to radial distance to tumor margin—all distances are expressed as a fraction indexed to the margin. The default is the normalized distance from the centroid to the margin and is represented from 0 to 1 step by 0.1. Consequently, in this embodiment there are 11 data points in every direction. However, an operator has the ability to adjust the number of data points if desired.

Geometric Direction:

The default number of directions is from 0 degrees to 357 degrees by 3 degree steps. In the same manner as the geometric distance, the number of geometric direction can be adjusted by the operator if desired.

Co-registration:

The values of each of the data points in the MRI image of the tumor with the same geometric distance and direction are averaged. Parametric maps are interpolated and co-registered.

Visual representation:

In the embodiment disclosed, each voxel in the plot is given 5 parameter values (normalized radial distance, angular distance, nCBV, kTrans, 1/ADC). The voxels in the X-Y plane are based on the normalized radial distance and the angular distance. The height of a voxel in the +Z direction is encoded with the normalized cerebral blood volume (nCBV) parameter. A height of a voxel in the −Z direction is encoded with kTrans*10. The color of the voxels in the plot is mapped to 1/ADC value (scaled to 1×10e−3 mm²/s). It is very important to select the same range of each value for a visual comparison between each grade of tumors.

The plot of the parameters described above creates a standardized “tumor space” that facilitates intuitive understanding of multiple tumor attributes by the radiologists, clinicians and any other individual involved in treating or understanding tumor biology. In addition, the plot facilitates qualitative comparisons between tumors by summating multiple kinds of parametric data into a single image. Moreover, this technique also allows quantitative comparisons and averaging using the imaging attributes of multiple tumors, facilitating automated and semi-automated tumor analysis, biological behavior prediction and histological identification.

By virtualizing tumors into mathematically distinct representations, enabling averaging and partitioning into distinct groupings (independent of histologic grade and standard imaging characteristics), similar histologic grade tumor may be further defined into those responsive to a given chemotherapy/radiation regimen, likelihood of recurrence or other important clinical parameter.

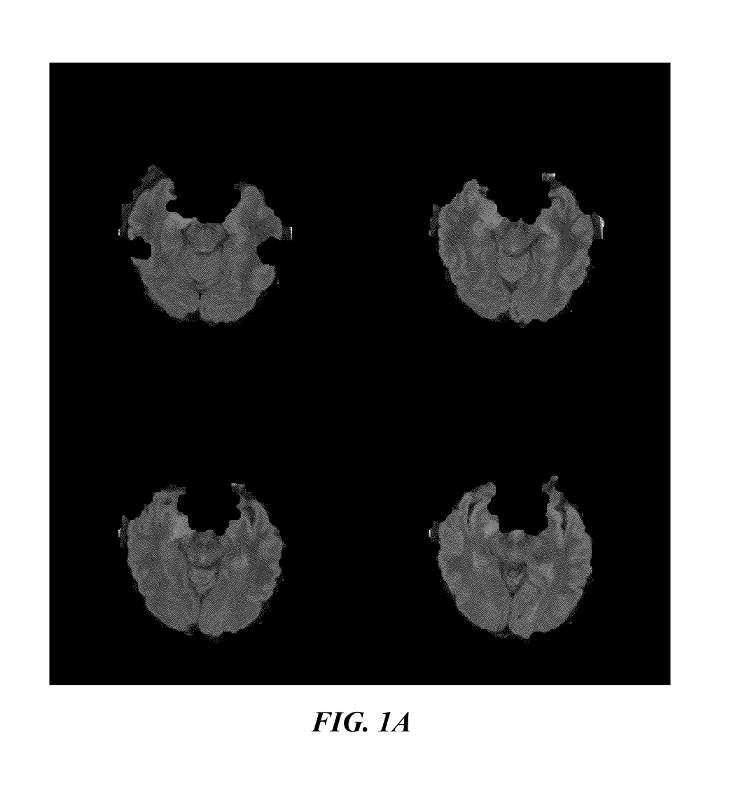

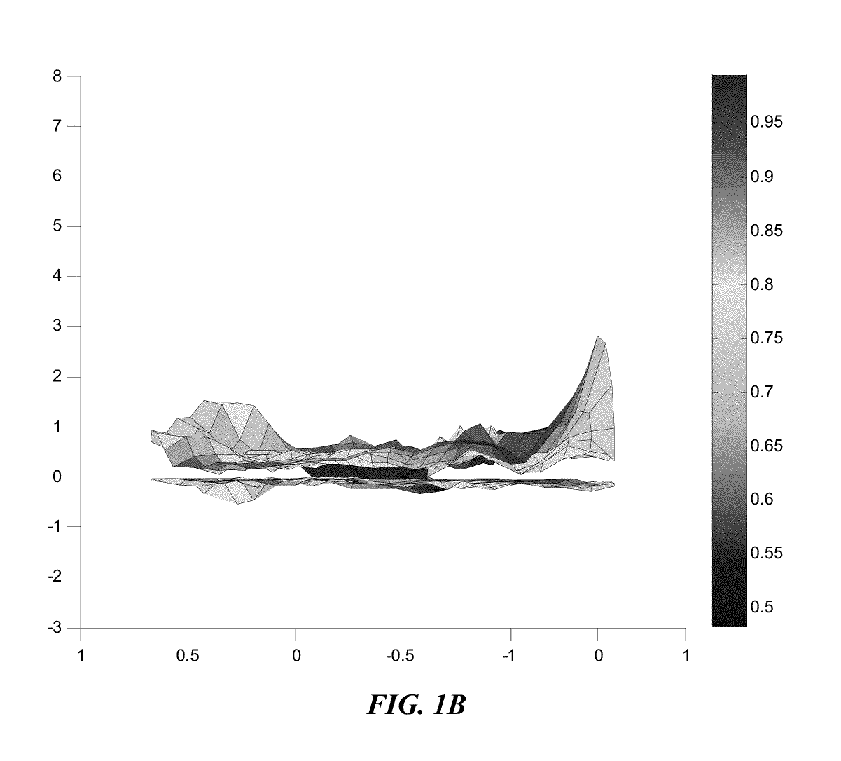

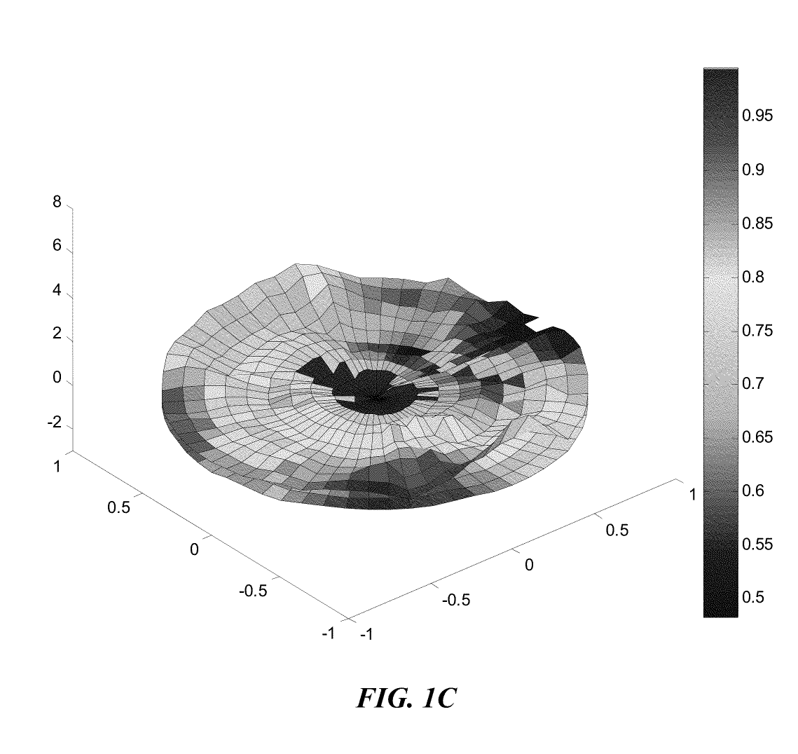

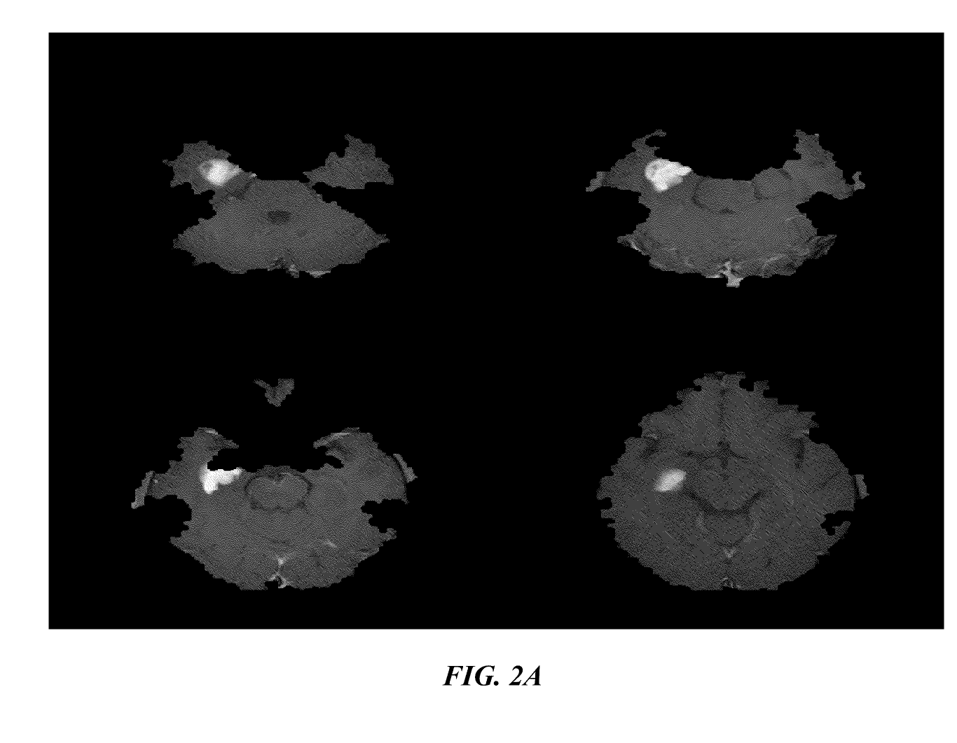

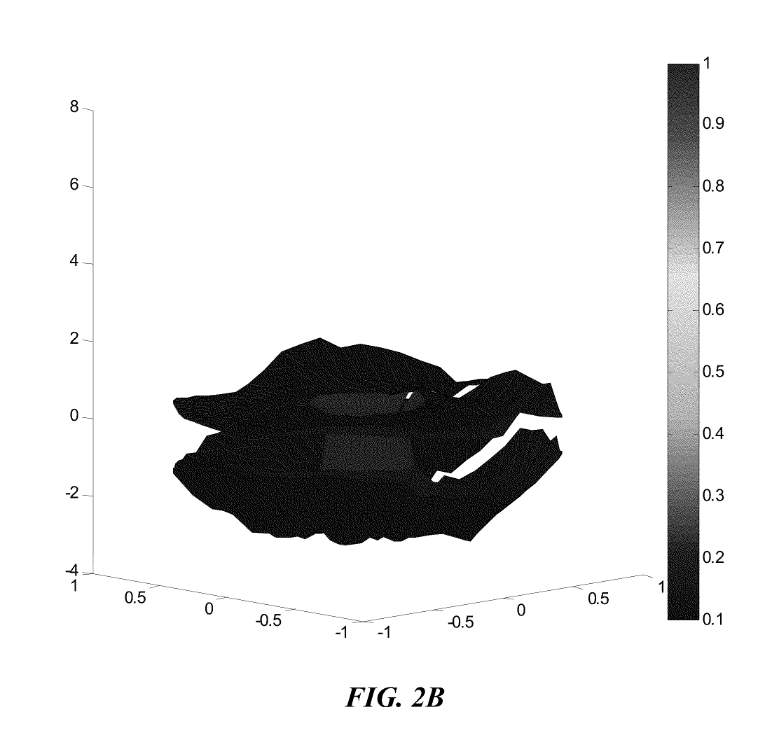

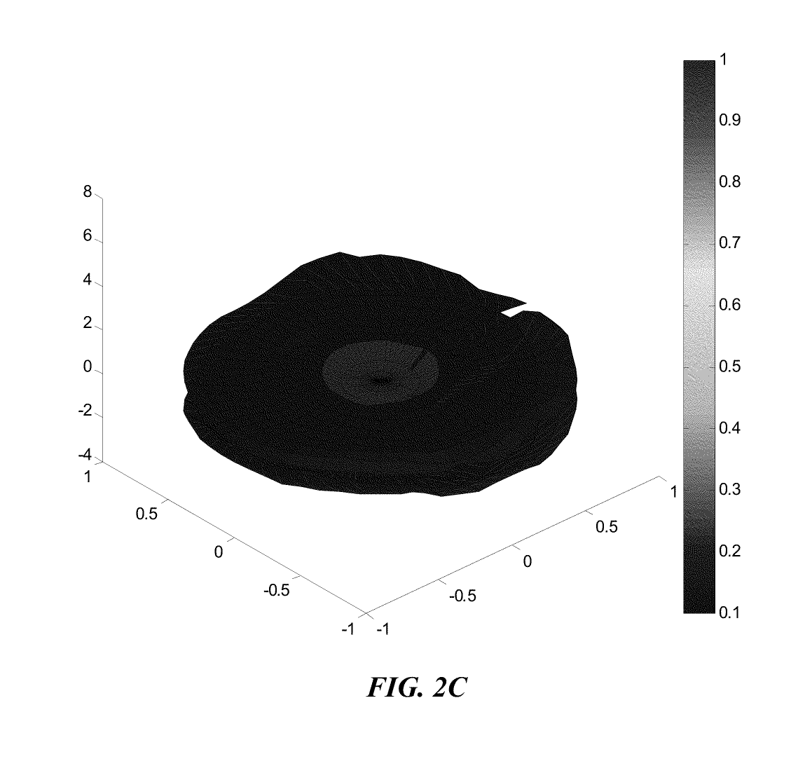

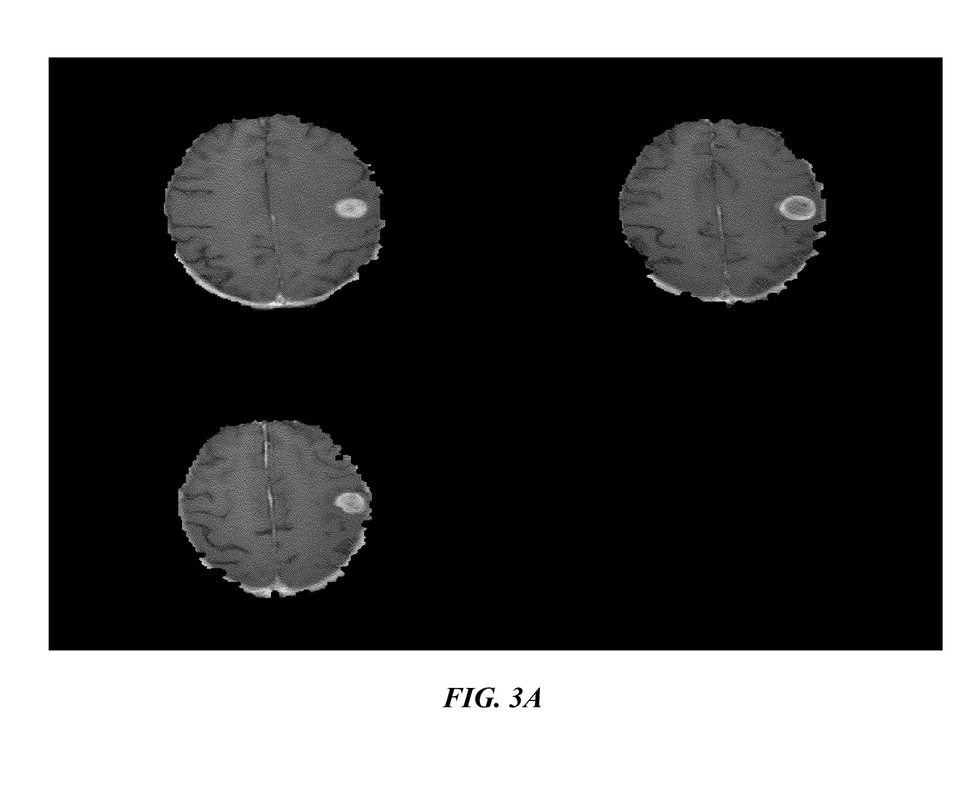

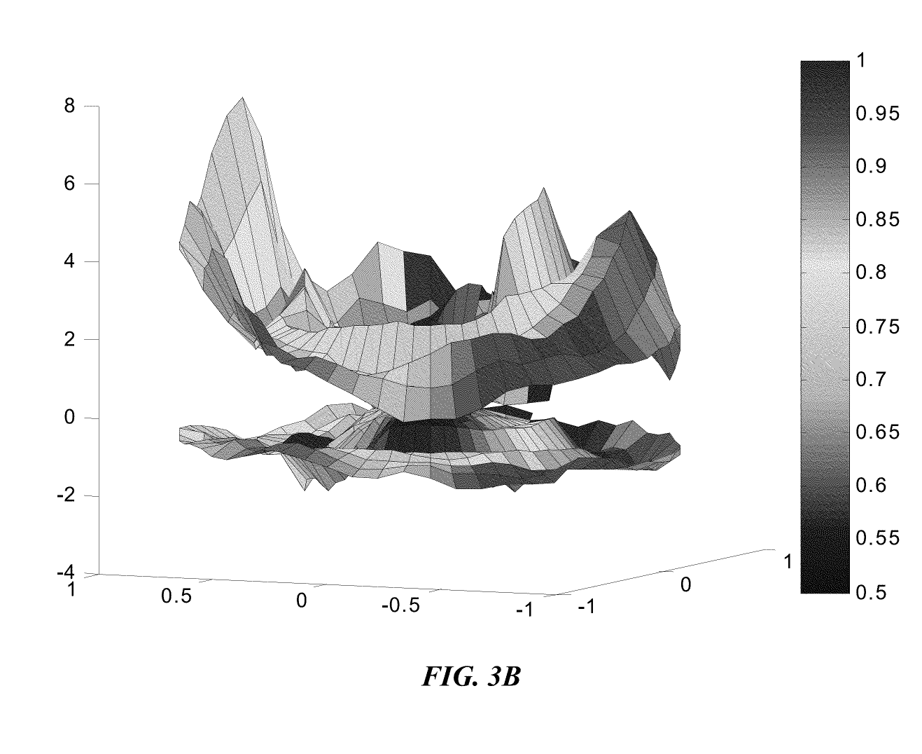

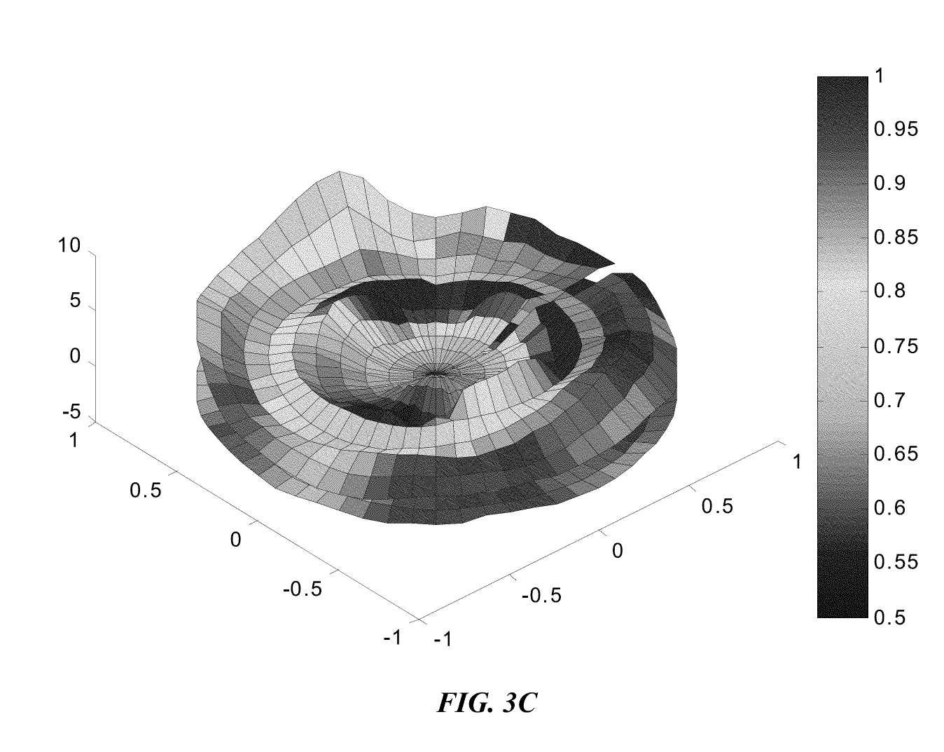

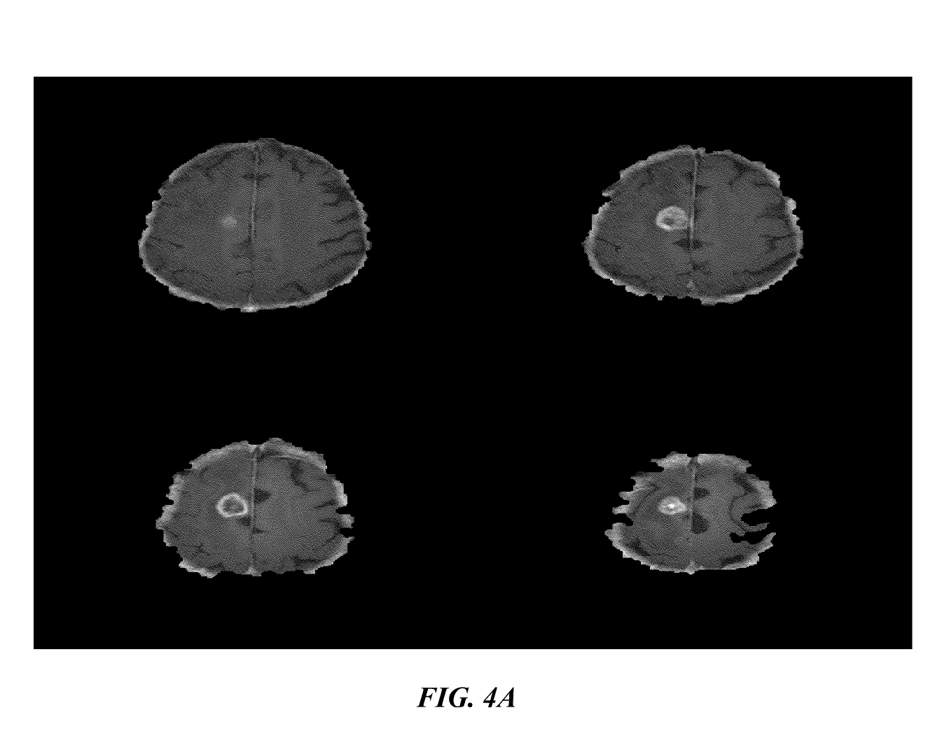

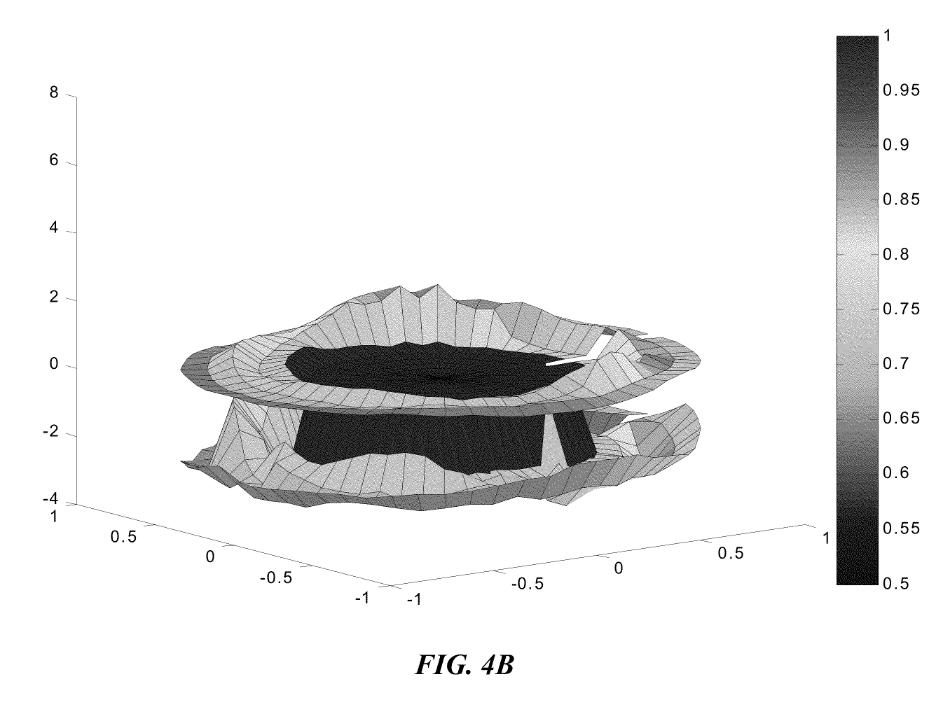

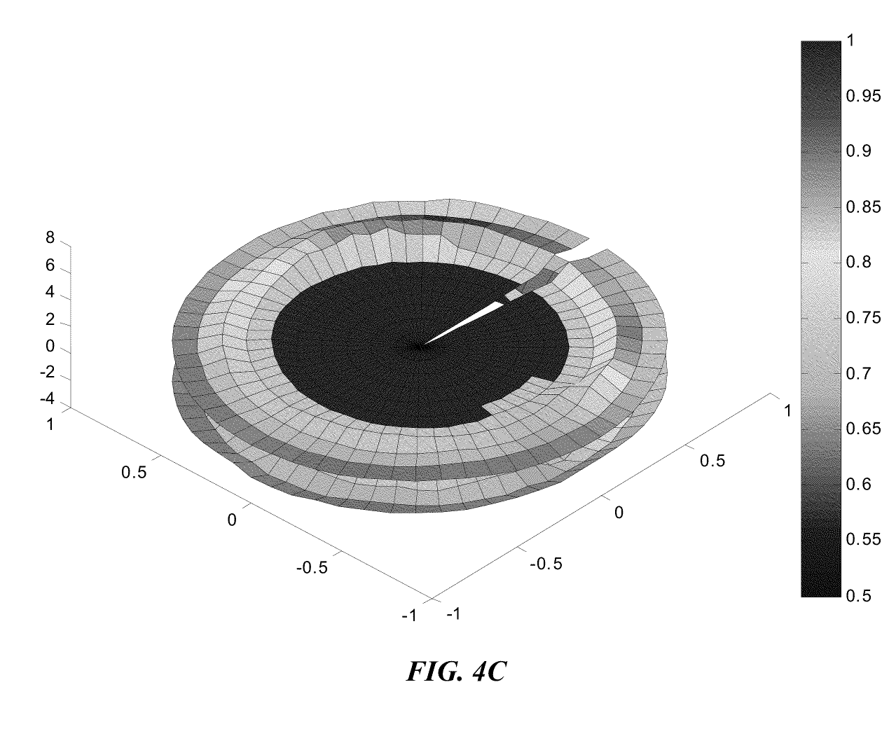

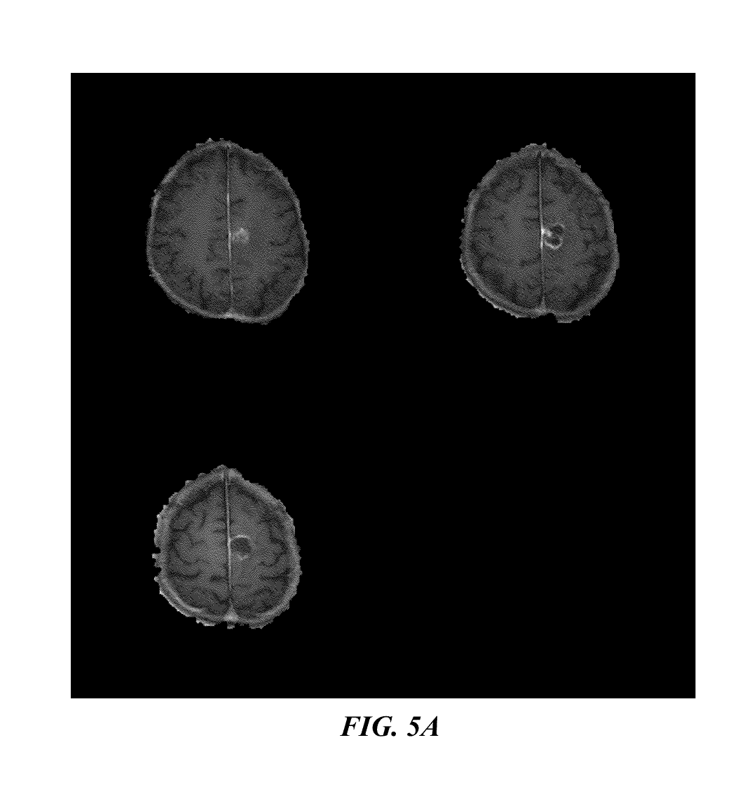

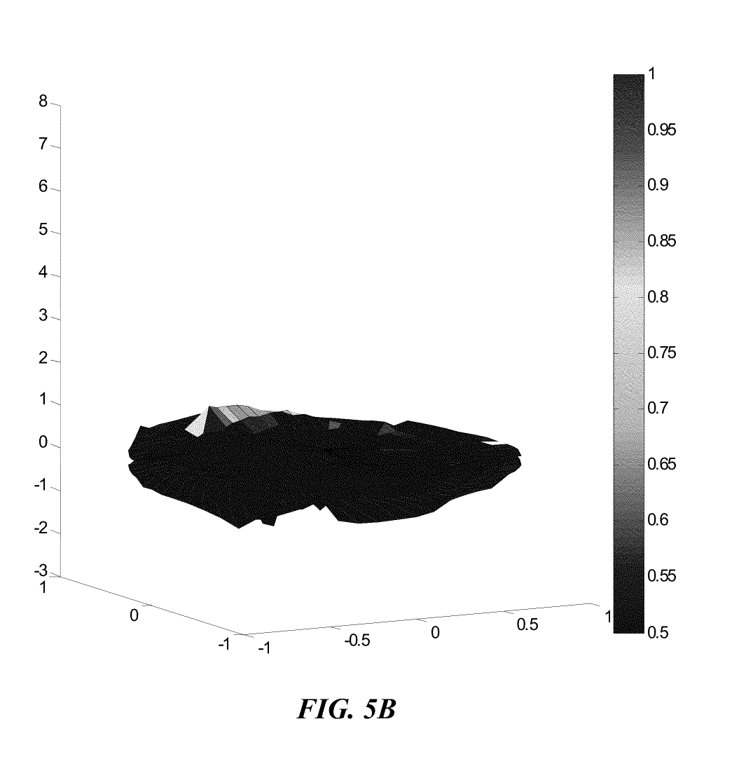

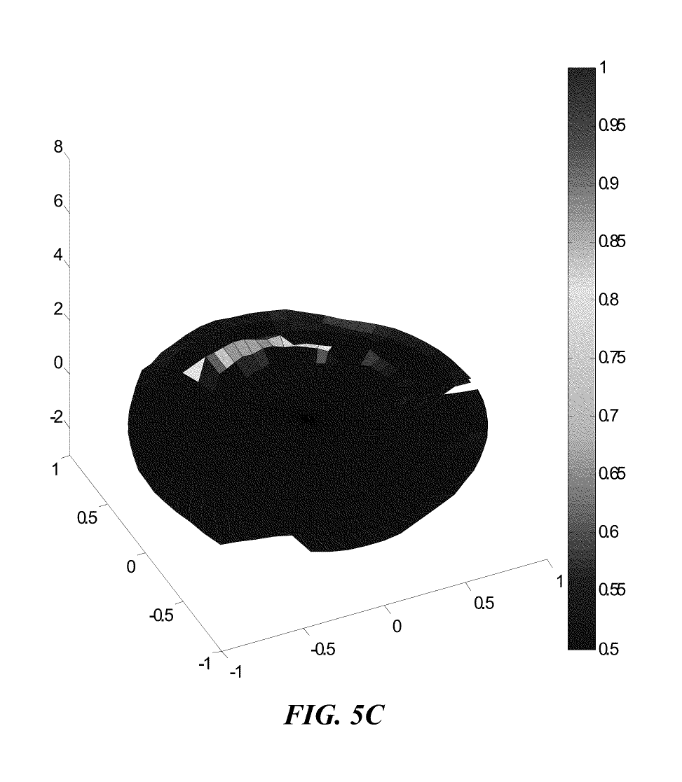

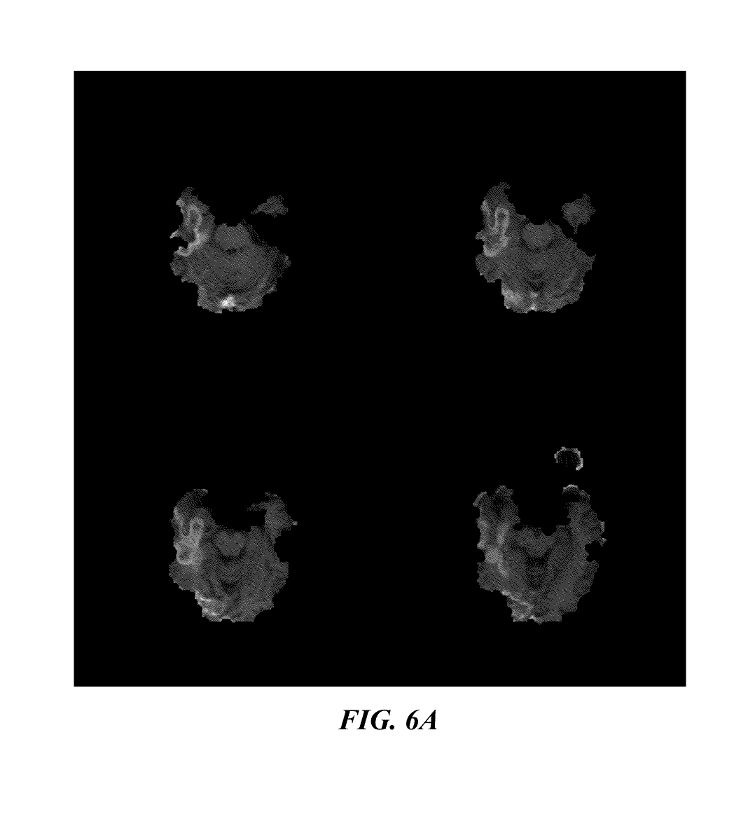

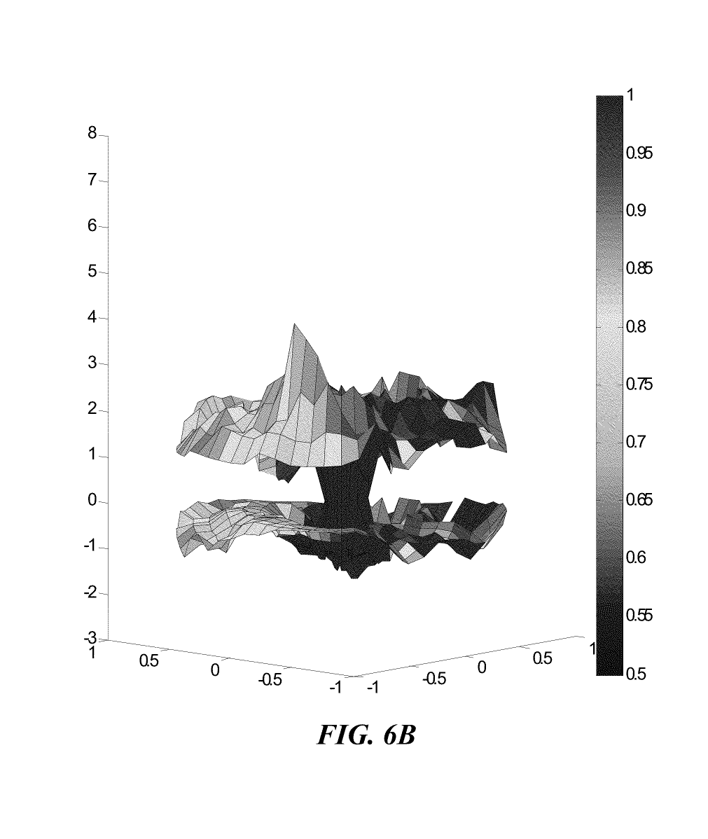

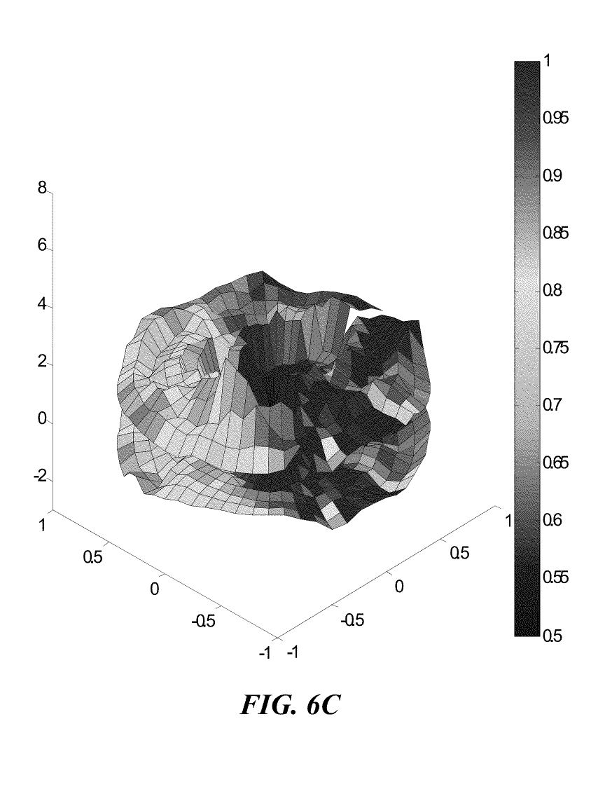

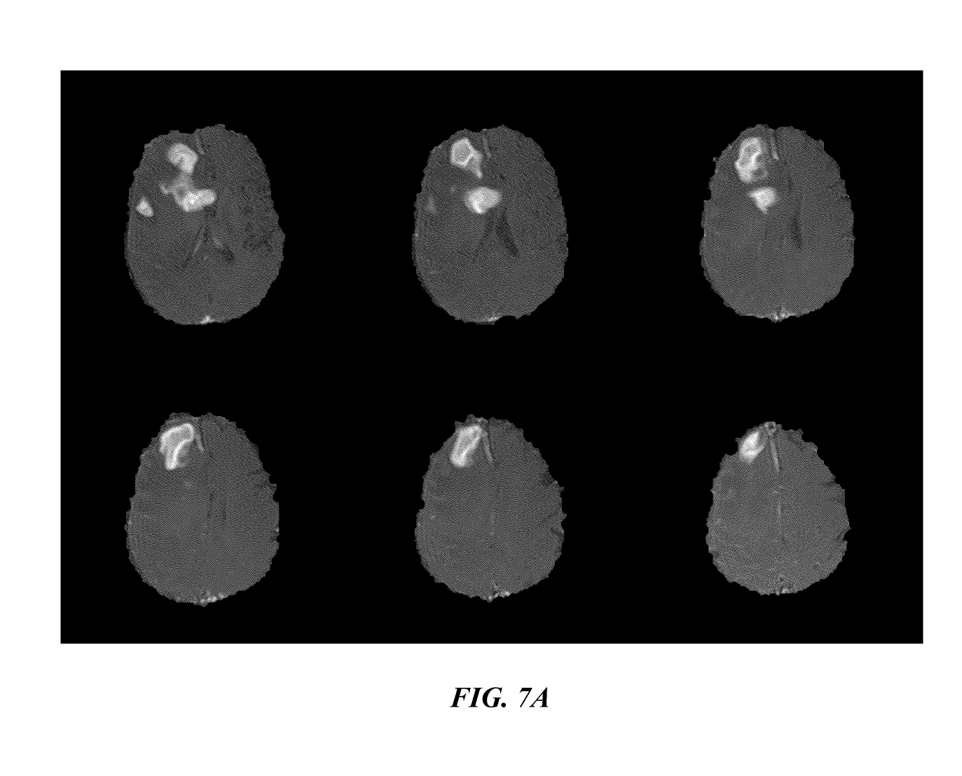

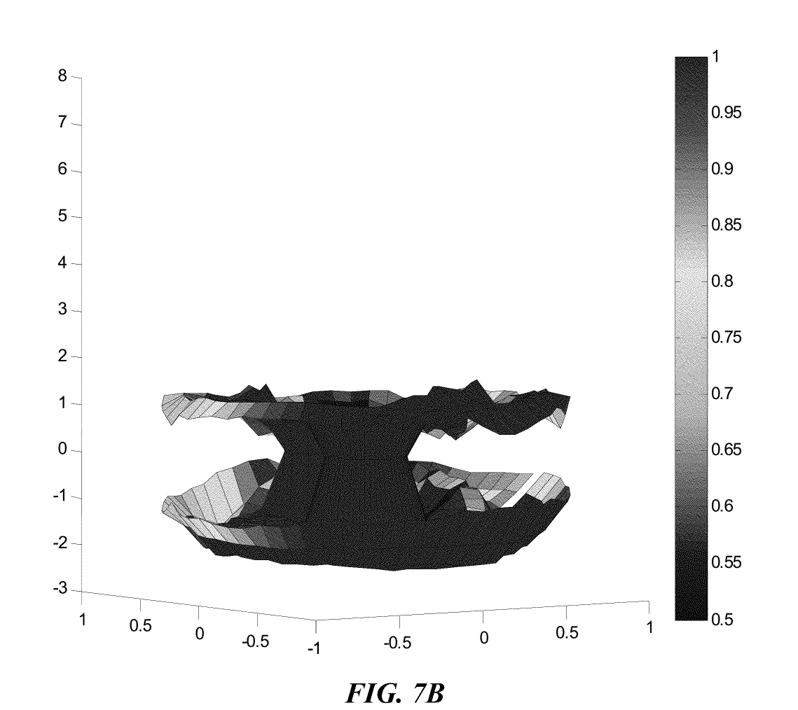

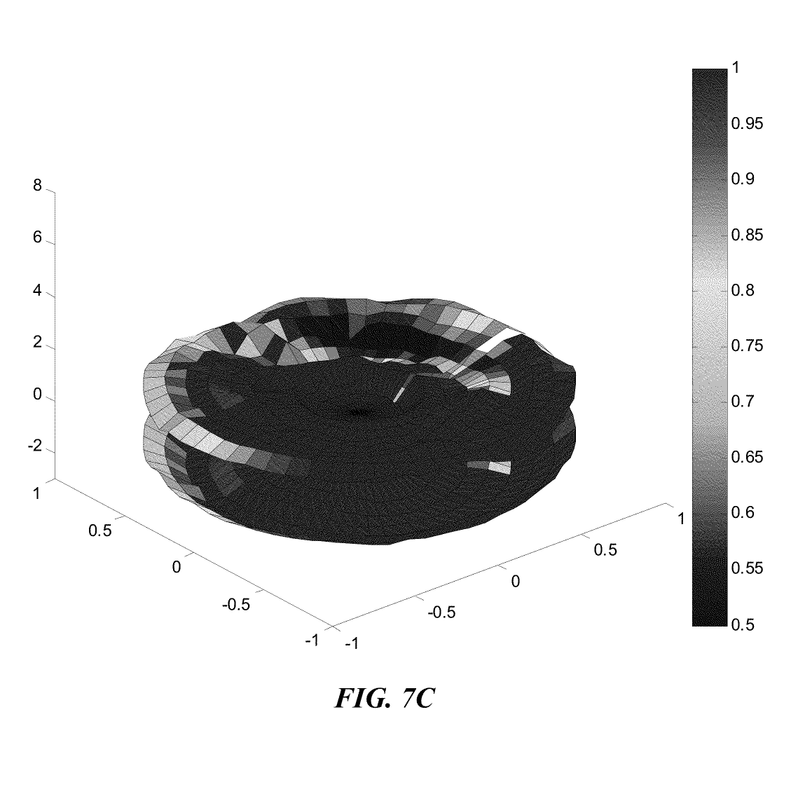

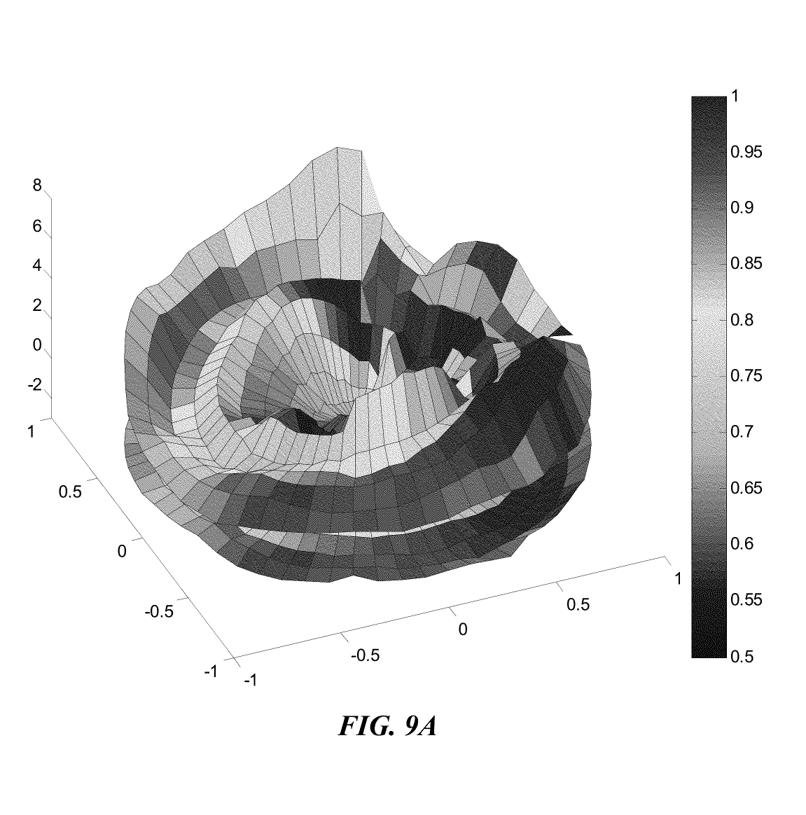

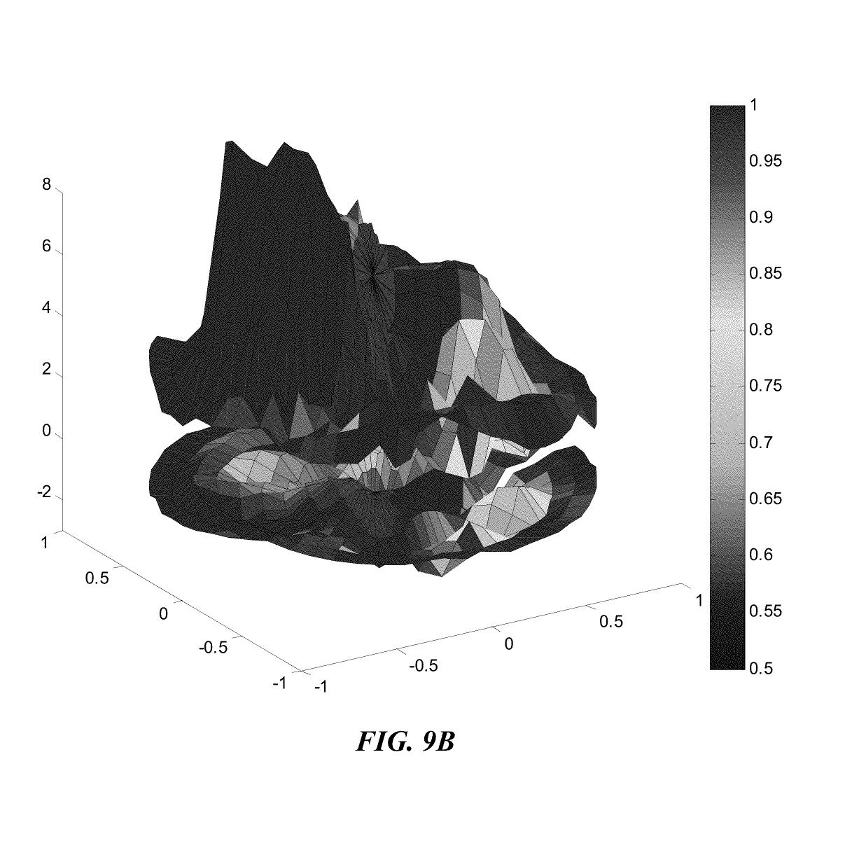

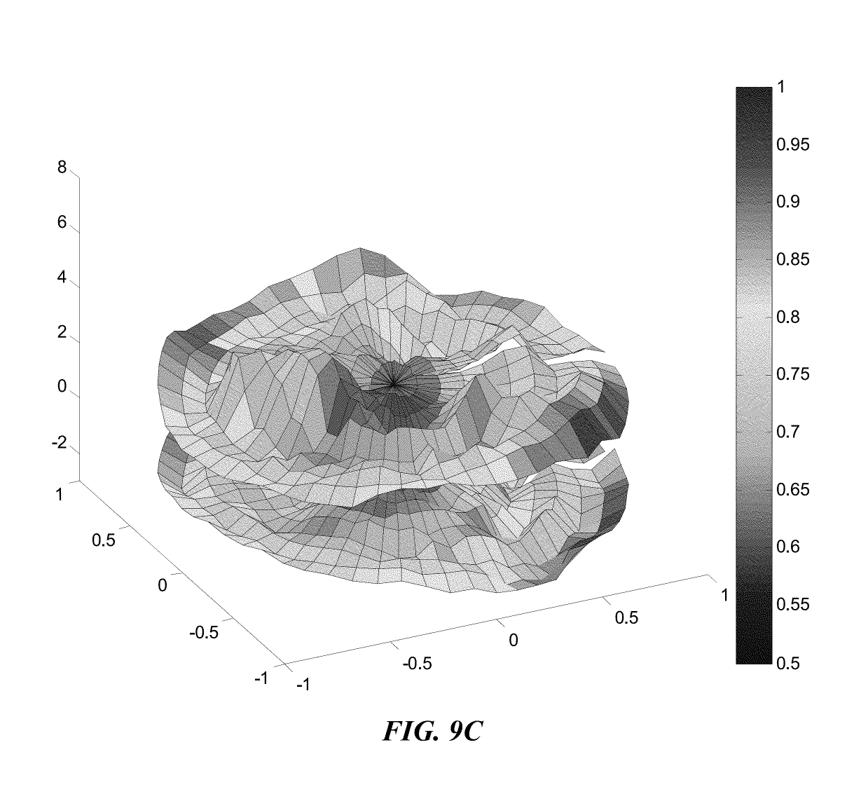

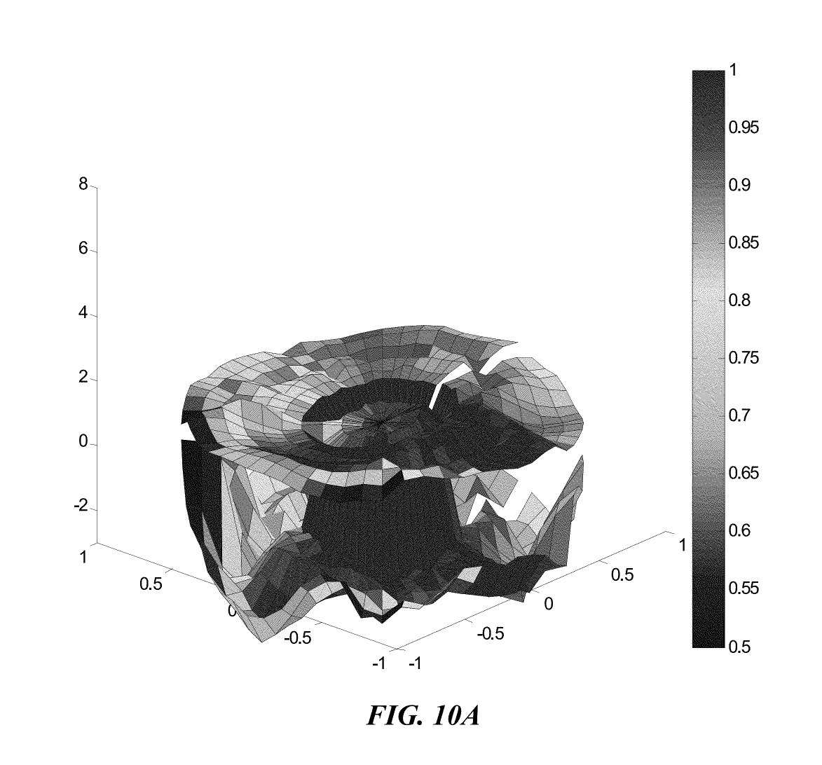

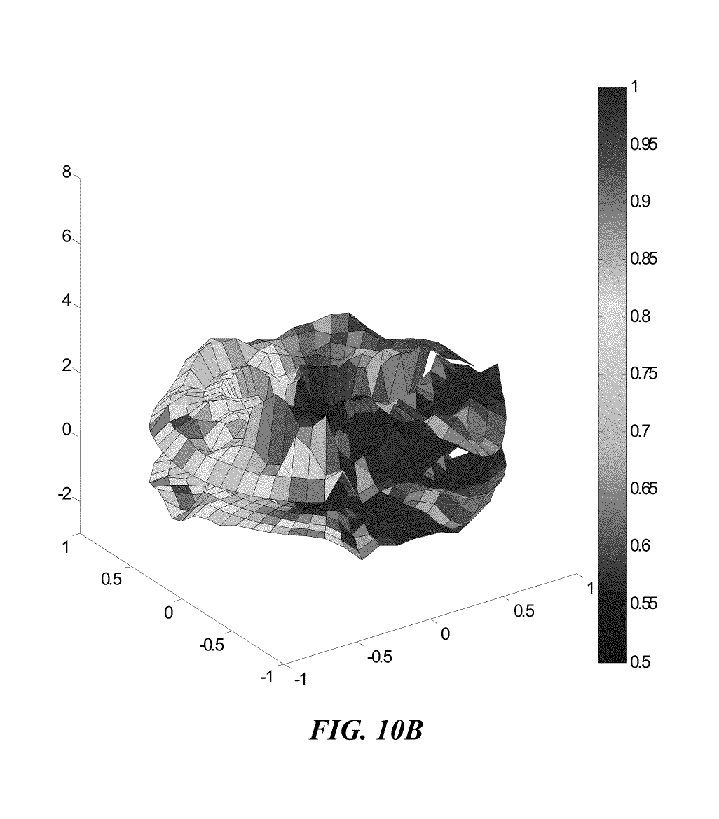

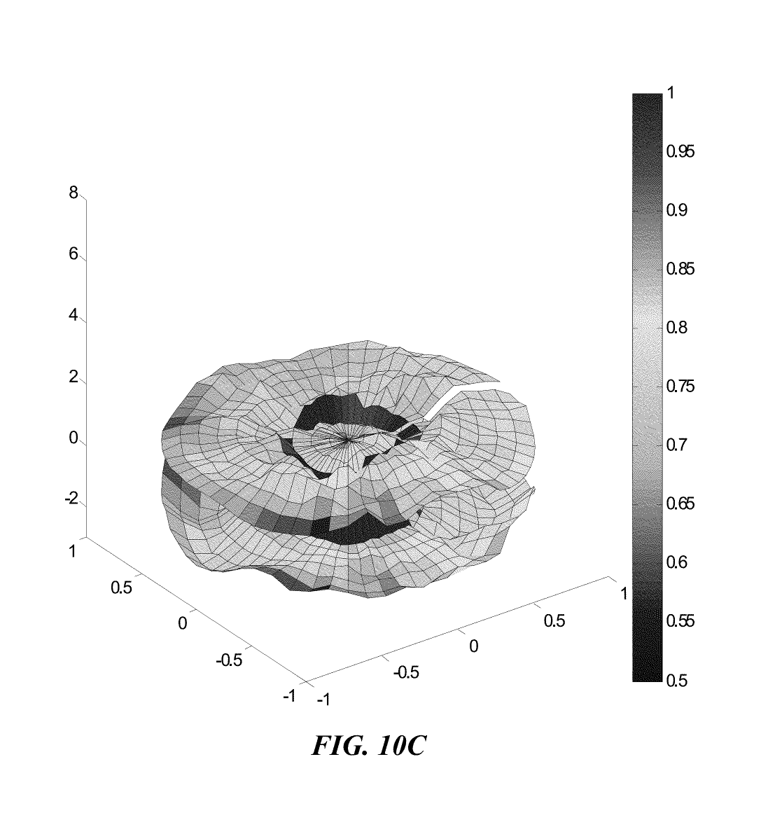

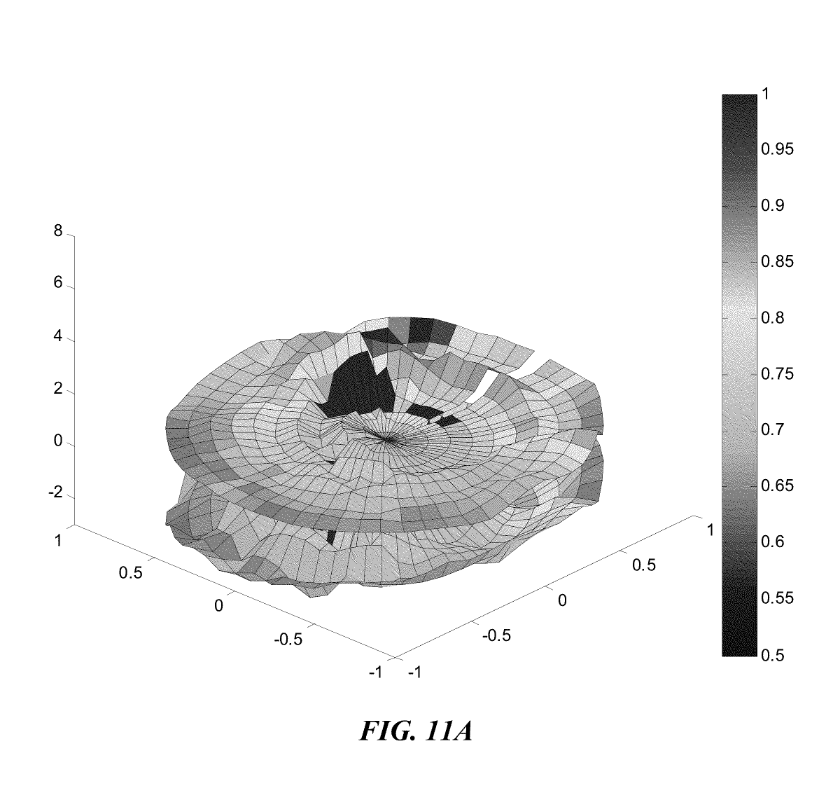

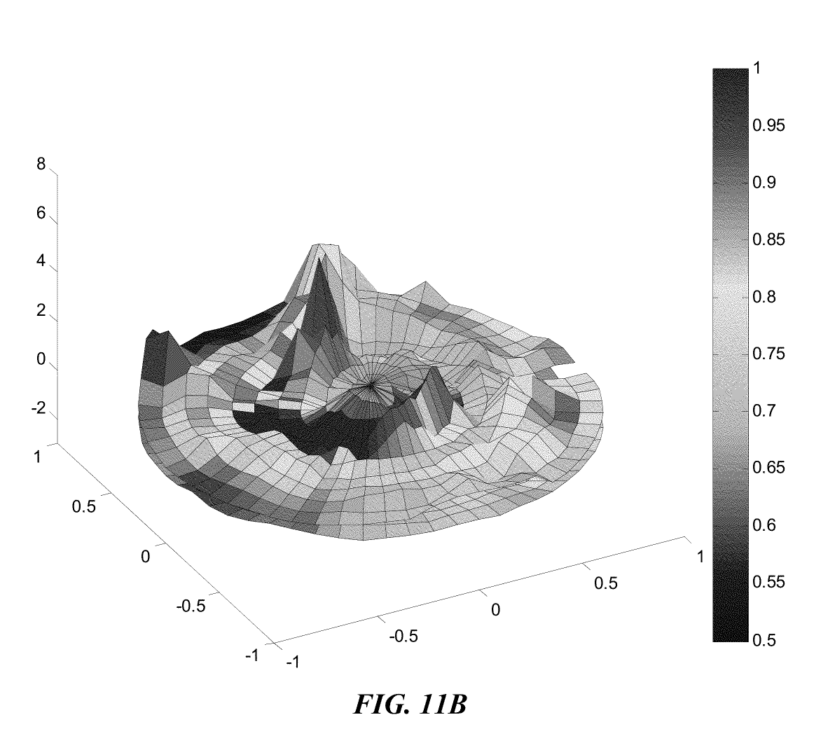

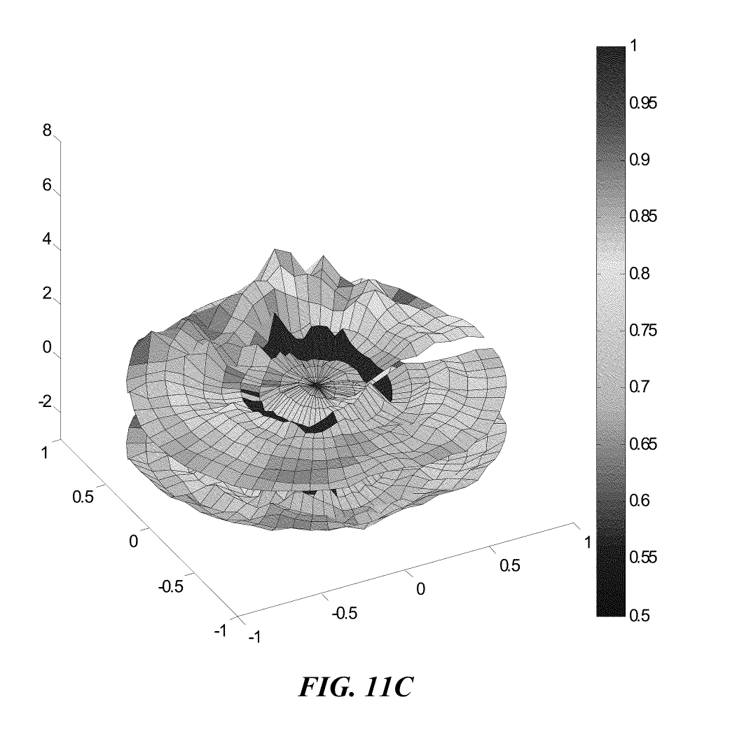

FIGS. 1-11 are examples of the virtualization of multiple tumors.

FIGS. 1-7, parts A show the anatomic images of the tumors, demonstrating variability in size, location and shape. Parts B and C show the virtualization of these multiple tumors in the single standardized tumor space that is here disclosed.

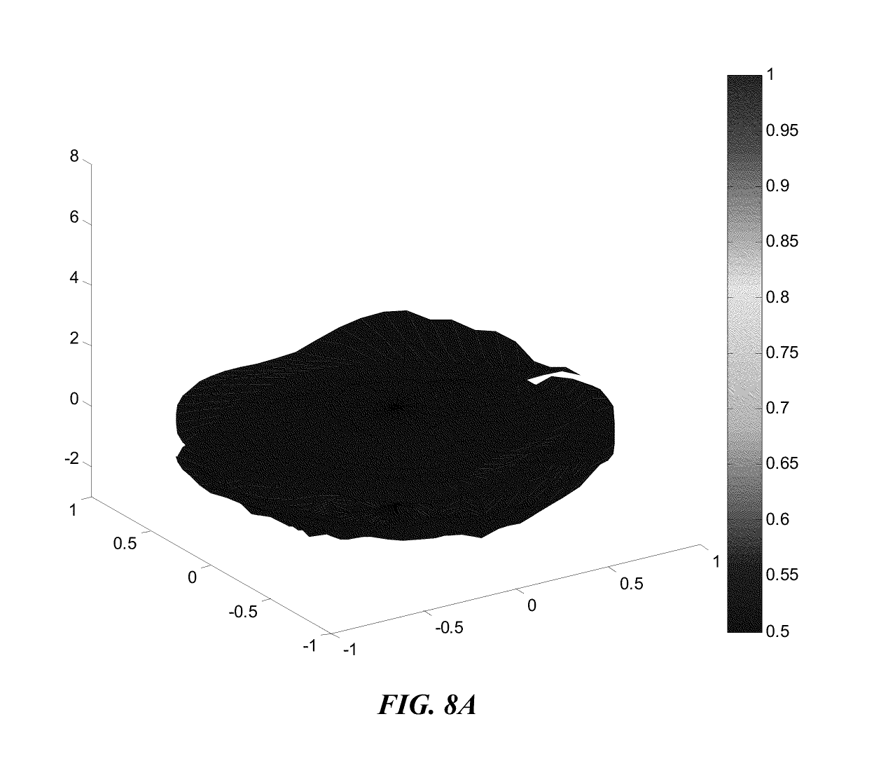

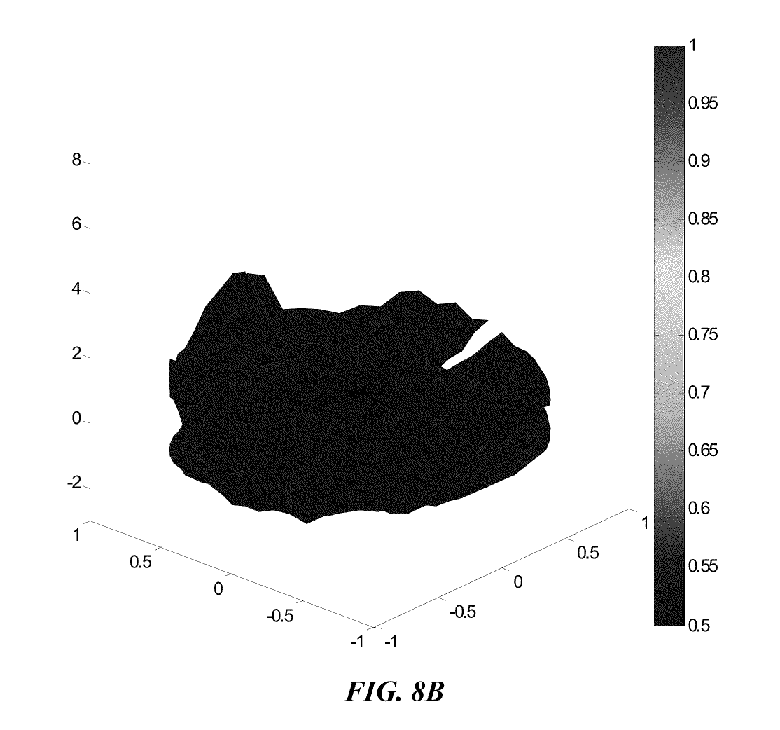

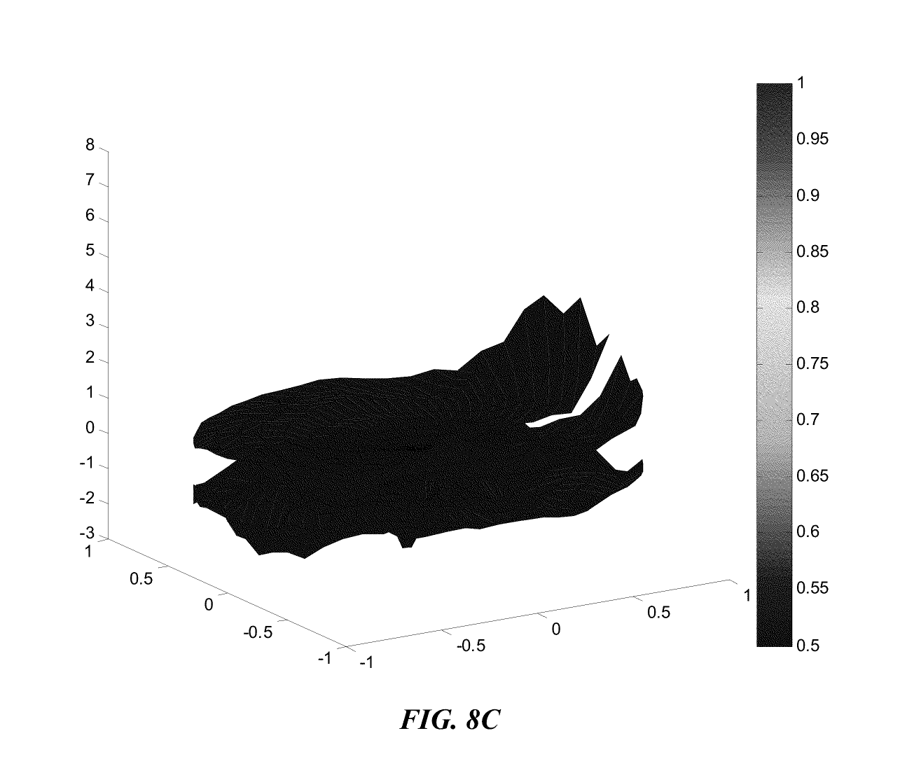

FIGS. 8-11, parts A and B show examples of two tumors of a similar histological grade and type (demonstrating reproducibility of the technique). Part C shows the mathematical average of the examples from parts A and B, demonstrating the capacity to perform mathematical functions and analysis on the virtualized tumors—as a consequence of the approach disclosed here. Mathematical analysis and transforms are possible on multiple tumors—only because we have abstracted the tumors into the standardized space disclose here.

No smoothing algorithm is applied (although one could easily be added), accounting for the “jagged” appearance. This aspect specifically points out the heterogeneity of the tumor—where there are substantial biological differences within a single tumor. Also notable, this method shows the entire tumor as a single visualizable object.

Because the attributes of the representation are independently calculated, and a tumor is quite heterogenous—a single point in the tumor have not have all aspects (variables) with the same relationship (i.e. there is not necessarily high internal correlation between variables).

From the foregoing, it will be appreciated that specific embodiments of the invention have been described herein for purposes of illustration, but that various modifications may be made without deviating from the scope of the invention. Accordingly, the invention is not limited except as by the appended claims.

I/we claim:

1. A medical imaging system, comprising
a processor configured to receive MRI data from an MRI image of a tumor and determine parameters from data points in the MRI image including a centroid of the tumor, a normalized cerebral blood volume (nCBV), a contrast transfer rate constant (kTrans) and an apparent diffusion coefficient (ADC) that is calculated from Bo and B1000 MRI images for data points in the MRI data,
wherein the processor is further configured to produce a plot of the MRI parameters, where the plot has a number of voxels each representing one or more corresponding data points in the MRI image, wherein each voxels is positioned in an X-Y plane of a three dimensional plot at a distance representing a normalized distance of the corresponding data points to a maximum diameter of the tumor and at an angle representing the angle of the corresponding data points with respect to the centroid of the tumor and wherein the nCBV, kTrans and 1/ADC are represented by the height of the voxel in one of the +Z or −Z directions on the three dimensional plot and the color of the voxel.

2. The system of claim 1, wherein nCBV is mapped to a voxel as a varying height in the +Z direction.

3. The system of claim 1, wherein kTrans is mapped to a voxel as a varying height in the −Z direction.

4. The system of claim 1, wherein 1/ADC is mapped to a voxel as a varying color in the plot.

5. A method of operating medical imaging system, comprising
receiving MRI data from an MRI image of a tumor at a processor and determining with the processor parameters from data points in the MRI image including a centroid of the tumor, a normalized cerebral blood volume (nCBV), a contrast transfer rate constant (kTrans) and apparent diffusion coefficient (ADC) calculated from Bo and B1000 MRI images for data points in the MRI data; and
producing a plot of the MRI parameters with the processor, where the plot has a number of voxels each representing one or more corresponding data points in the MRI image, wherein each voxels is positioned in an X-Y plane of a three dimensional plot at a distance representing a normalized distance of the corresponding data points to a maximum diameter of the tumor and at an angle representing the angle of the corresponding data points with respect to the centroid and wherein the nCBV, kTrans and 1/ADC are represented by the height of the voxel in one of the +Z, −Z direction on the three dimensional plot and a color of the voxel.

6. A non-transitory computer readable media including instructions that are executable by a processor in a medical imaging system to cause the processor to:
receive MRI data from an MRI image of a tumor and determine parameters from data points in the MRI image including a centroid of the tumor, a normalized cerebral blood volume (nCBV), a contrast transfer rate constant (kTrans) and apparent diffusion coefficient (ADC) calculated from Bo and B1000 MRI images for data points in the MRI data,
produce a plot of the MRI parameters with the processor, where the plot has a number of voxels each representing one or more corresponding data points in the MRI image, wherein each voxels is positioned in an X-Y plane of a three dimensional plot at a distance representing a normalized distance of the corresponding data points to a maximum diameter of the tumor and at an angle representing the angle of the corresponding data points with respect to the centroid and wherein the nCBV, kTrans and 1/ADC are represented by the height of the voxel in one of the +Z, −Z direction on the three dimensional plot and a color of the voxel.

7. A non-transitory computer readable media containing instructions that are executable by a computer in a medical imaging system to:
receive a number of non-anatomic parameters computed from imaged locations in a tumor;
plot the non-anatomic parameters from the imaged locations in a normalized multi-dimensional space; wherein imaged locations in the tumor are represented by voxels that are shown at normalized locations in the multi-dimensional space and are encoded with at least two non-anatomic parameters.
